(19) United States
(12) Patent Application Publication
(10) Pub. No.: US 2016/0324680 A1
(43) Pub. Date: Nov. 10, 2016

(54) Method for the Opening of Airway by the Use of a Soft Cervical Collar

(71) Applicant: Hospital Universitario San Ignacio, Bogota (CO)

(72) Inventors: Jheremy Enrique Reyes Castellanos, Bogota (CO); Alvaro de Jesus Ruiz Morales, Bogota (CO); Patricia Hidalgo Martinez, Bogota (CO)

(21) Appl. No.: 14/706,563

(22) Filed: May 7, 2015

(51) Int. Cl. A61F 5/56

(52) U.S. Cl. CPC A61F 5/56

(57) ABSTRACT

The present invention belongs to the field of medicine and relates to a method for the treatment of the obstruction of the airway, specially moderated obstructive sleep apnea/hypopnea syndrome (OSAHS) in adult patients, by using a soft cervical collar. The method of the invention achieves a significant reduction of the apnea/hypopnea index (AHI) in adult patients diagnosed with obstructive sleep apnea of moderate level.

### FIELD OF THE INVENTION

Method for non-surgical treatment of respiratory diseases by using an orthopedic equipment.

### CLASSIFICATION OF THE INVENTION

International Patent Classification: Orthopedic apparatus of cervical collar type allowing non-surgical treatment of bones and joints (A61F 5/55).

U.S. Classification: 128/848 (antisnoring devices), 602/92 (antisnoring or mouth dosing devices); 602/18 (splint, brace, or bandage for neck).

### BACKGROUND OF THE INVENTION

Obstructive sleep apnea is a medical condition produced by the displacement of soft tissue and muscle relaxation in the posterior area of the throat that interrupts air intake and normal breathing, causing interrupted sleep periods for the patient. This repeated interruption in the airflow intake through nose and mouth is produced during at least 10 seconds and produces an oxygen desaturation of at least 10% (Ingbar and Lee, 1985). The person suffering of obstructive sleep apnea presents symptoms such as daytime sleepiness, sleep attacks, unusual snoring or gasping during sleep, headaches, tiredness and chronic fatigue, as well as respiratory alterations, cardiac arrhythmia and arterial hypertension during sleep (Vgontzas and Kales, 1999; US2012/0234330, Saiz).

Obstructive sleep apnea syndrome is defined by the presence of an apnea/hypopnea index (AHI) greater than 5 in adult patients. This index is calculated in the following manner: apnea/hour index=(Total number of apneas×60)/total sleeping time. The importance of this index lies on the fact that, based on its results, diagnosis and later treatment guides are established that could be classified as: mild (AHI 5-15), moderate (AHI: 15-30) or severe (AHI: greater than 30).

Different risk factors for suffering of obstructive sleep apnea have been detected, including obesity, gender, cervical size, craniofacial malformations and heredity. Currently, the treatment against obstructive sleep apnea is based on keeping the airway clear, having a good sleep hygiene (habits/schedule), reducing weight (recommended in obese or overweight patients), as well as including modification of risk factors, using intraoral devices, surgical treatment in recommended cases, pharmacological treatment if necessary, and/or Continuous Positive Airway Pressure—CPAP, which currently is the first-line treatment.

In effect, a suitable CPAP therapy can prevent any obstruction degree of the upper airway in all sleeping phases and body positions, in order to restore normal sleep. However, even though CPAP therapy is suitable for the treatment of apnea, its implementation by the patient is not simple given that it requires a comprehensive educational therapy for the patient and relatives, in such a way that they accept and adapt to the treatment.

In addition to the above, sometimes CPAP therapy produces adverse effects such as rhinitis, conjunctivitis and local cutaneous eruptions. On the other hand, implementation of the treatment is difficult due to the cost associated to the equipment and maintenance supplies. But in general, the reasons for the failure of CPAP use are related to the difficult of the patient for adapting to the treatment, given that the use of the device could result uncomfortable for the patient and/or the room partner. As a consequence of the above, about 30% of sleep apnea patients do not accept this treatment, while remaining patients accept the treatment but do not use it through all the night.

Another alternative treatment form used for apnea is the use of orthopedic devices of the cervical support-type, which must be used by the patient through all the night in order to maintain a suitable posture and stretching of the neck. In this sense, some cervical devices designed for treating apnea are known in the state of the art. For instance, patent document U.S. Pat. No. 4,700,697 discloses the use of a complex device consisting of an anterior support column with a chin cup and a rigid scutum, as well as a bandage that surrounds the neck of the individual and avoids ventral flexion.

In turn, patent document U.S. Pat. No. 6,171,314 discloses the use of a cervical device with central support and rigid side supports, wherein said supports include a chin contact surface and body contacting surfaces that are rigid with the purpose of avoiding ventral and lateral neck movement.

Likewise, patent documents U.S. Pat. No. 7,789,843 discloses the use of a helping operation device for elevation and forward thrusting of the jaw, keeping extended the neck of the individual in this manner. Said device comprises a rigid base contacting the torso and another rigid base contacting the chin of the individual.

All of these devices comprise rigid or semi-rigid support structures and look for immobilize the neck of the patient and restrict its movements making them uncomfortable to be used during the night and preventing adaptation to different positions of the patient during sleep.

In turn, document US 2010/0234330 relates to a system and method for treating sleep apnea and snoring based on the use of a removable collar with a tubular sleeve inside made of foam rubber that is divided into three segments having sphere-like elements on the inside. In agreement with the inventor, said device forces the head to remain in upright or extended position and the muscle tissues of the throat to stay tight, allowing in this manner the airflow in the throat region. However, the division of the collar into three sections makes that location of the collar must be precise and that the slipping of the collar during sleep may cause discomfort to the patient.

Therefore, there is a persistent need to have a comfortable device to be used during the night, that does not totally restrict the movements of the head and that keeps the muscles of the throat extended so that the obstruction of the airway during sleep is decreased and a reduction of snoring and apnea episodes is achieved.

### GENERAL DESCRIPTION OF THE INVENTION

The present invention is addressed to a method for opening the airway of a human subject. The method includes placing a soft orthopedic device in the neck of the subject in order to stretch and rectify the airway, so that it can offer an anatomical and functional improvement to obstructive sleep apnea of moderate type.

Another aspect of the present invention relates to the use of a soft cervical collar that lacks of rigid or semi-rigid supports, made of medium density foam, for the treatment of moderate obstructive sleep apnea.

In yet another aspect of the invention it relates to the use of the Thomas-type collar for the treatment of moderate obstructive sleep apnea.

### DETAILED DESCRIPTION

The method for opening the airway according to the present invention, primarily addressed to the treatment of moderate obstructive sleep apnea, consists of placing a removable cervical collar in the neck of the patient, which remains in position during the sleeping period with the aim of achieving the stretching of the muscles of the throat and rectification of the airway. In this way, the upper airway remains clear, the airflow is continuous and the obstruction of the airway due to wrong postures and/or to muscular obstruction of the hypoglossal muscle is reduced.

This collar is made of medium density foam, has the shape of a band with ends slightly narrower than the rest of the body of the collar, has a variable height that is adjusted to the height of the neck of the subject and has a Velcro-type strip to adjust according to the measure of the contour of the neck.

Due to its soft composition and the absence of rigid or semi-rigid supporting elements, the collar used in the present invention allows a comfortable adjustment and ensures that the neck remains in an extended position during sleep, but without completely restricting lateral or anteroposterior head movements.

In a preferred embodiment, the method for opening the airway according to the present invention consists of placing a Thomas collar in the neck of the patient, which remains placed during the sleeping period with the purpose of achieving stretching of the throat muscles and rectification of the airway.

The method of treatment of the present invention allows a decrease in the apnea episodes during sleep, decreasing snoring additionally, which finally produces a reduction in the apnea/hypopnea index (AHI).

The use of the soft cervical collar that lacks of rigid or semi-rigid supporting elements, allows the subject to place himself or herself in lateral position or in supine position during sleep. Likewise, the soft consistency of this collar avoids the subject from presenting lacerations, hematomas, or marks that are commonly produced by the use of cervical collars having rigid or semi-rigid structures.

The use of the soft collar that has a breathable material in contact with the skin avoids the production of an excessive heat or heat in the neck region due to the use of the cervical collar during the night allowing greater comfort for the subject under treatment.

Other uses of the soft cervical collar for treating apnea that consider, for instance, the use of the collar in determined sleep phases (e.g. in REM phase, or non-REM phase), or during partial sleeping periods (e.g. at the beginning of the rest period, or at the end of the rest period), are within the scope of the present invention.

According to the present invention, “moderate obstructive sleep apnea” is defined as the presence of an apnea-hypopnea/hour index (AHI) between 15 and 30.

In turn, a “soft orthopedic device” is understood as an orthopedic element that is placed in the neck and that lacks of rigid or semi-rigid elements. In a preferred embodiment of the invention, said soft orthopedic device is a Thomas collar.

The “rigid or semi-rigid elements” are defined as hard consistency elements that can have materials such as polyurethane, sheet, iron, plastic, wood, or the like.

### EXAMPLES

The following examples illustrate the use of the soft cervical collar and the advantages associated to the method for the treatment of moderate obstructive sleep apnea according to the present invention.

### Example 1

A randomized crossover experimental clinical study was carried out, by comparing the impact on the basal apnea hypopnea index (basal AHI), i.e., the index of apnea episodes when the treatment with the neck device has not been done and the AHI with the use of the soft cervical collar (Thomas collar). In order to establish the AHI a total of 54 polysomnography studies were made in 27 patients with moderate OSAHS, who were monitored during all the night.

Gender distribution was of 16 women (59%) and 11 men (40%) individuals, having a study group without statistically significant differences at this regard (p=0.67). Weight variations were minimal having an average of 79 Kg for the women group and of 81 Kg for the men group.

Within the assessed parameters were included snoring ailment, sleepiness, fatigue, nocturnal awakenings ailment, cephalea ailment, and the presence of involuntary leg movements.

The apnea hypopnea index (AHI) without treatment was measured, finding a mean of 21.8 points which differentiated by gender was of AHI: 22 for the women group and AHI: 21 for the men group (Table 1), without significant differences between groups (p=0.74).

TABLE 1
Analysis of previous AHI and collar AHI

| | Number of | Average | Standard |
|---|---|---|---|
| | observations | AHI | deviation |
| Previous | 27 | 21.8 | 4.8 |
| AHI | | | |
| Collar AHI | 27 | 15.8 | 11.8 |
| Difference | 27 | 6.0 | 11.7 |

From the point of view of the reduction of AHI with the use of the collar (Table 2), it was found that the mean AHI with the use of the soft cervical collar was of 15.8 points (19.3 points for the women group and 13.3 points for the men group), maintaining the homogeneity between groups given that there were no significant differences in the gender comparison (p=0.81).

TABLE 2
Frequencies distribution of AHI/Collar by gender

| | Number of | | Standard |
|---|---|---|---|
| Gender | observations | Average | deviation |
| Feminine | 11 | 19.3 | 11.5 |
| Masculine | 16 | 13.4 | 11.8 |
| Total | 27 | 15.8 | 11.8 |

Regarding the evaluation of the reduction or not of AHI previous to treatment and subsequent to use of the soft cervical collar for the whole group, it was found an AHI reduction of 6.01 points (pre AHI: 21.89 vs. collar AHI: 15.87) (Table 2), which demonstrates the effectiveness of the cervical collar for treating moderate OSAHS.

It is observed from these results that the treatment with the soft cervical collar (Thomas collar) produces a decrease in the AHI values for both men and women.

### Example 2

With the same group of the example above, a rapid eye movement (REM) latency measure was made prior to the treatment and using the Thomas collar. Values are given in time in minutes from the beginning of the sleep until the first REM period (Table 3).

TABLE 3
Variation of REM latency with Collar

| | Number of | Average | Standard |
|---|---|---|---|
| | observations | (minutes) | deviation |
| Basal Latency | 27 | 101.4 | 52.3 |
| Collar Latency | 27 | 14.8 | 16.3 |
| Difference | 27 | 86.5 | 51.8 |

p < 0.0001

The results show a significant decrease in REM latency.

While the disclosure has been described in terms of exemplary aspects, those skilled in the art will recognize that the disclosure can be practiced with modifications in the spirit and scope of the appended claims. These examples given above are merely illustrative and are not meant to be an exhaustive list of all possible designs, aspects, applications or modifications of the disclosure.

What is claimed is:

1. A method for opening the airway of a human subject characterized in that a soft orthopedic device is placed in the neck of the subject during the sleeping period, where said device lacks of rigid or semi-rigid structures for achieving muscle stretching and airway rectification.

2. The method for opening the airway according to claim 1, characterized in that the orthopedic device is a soft cervical collar that is removable.

3. The method for opening the airway according to claim 1, characterized in that the soft orthopedic device is useful for the treatment of obstructive sleep apnea.

4. The method for opening the airway according to claim 3, characterized in that the obstructive sleep apnea is of moderate type.

5. A removable soft cervical collar that lacks of rigid or semi-rigid supporting elements characterized in that it is used for the treatment of moderate obstructive sleep apnea.

6. The soft cervical collar according to claim 5, which is a Thomas collar.

7. The soft cervical collar according to claim 5, characterized in that it is used for the treatment of moderate obstructive sleep apnea in adults.

8. The soft cervical collar according to claim 5 characterized in that it is used for the treatment of snoring.

9. The use of the soft cervical collar according to claim 5 for the treatment of moderate obstructive sleep apnea.
